(19) United States
(12) Patent Application Publication
(10) Pub. No.: US 2012/0016477 A1
(43) Pub. Date: Jan. 19, 2012

(54) LOCKABLE IMPLANT AND METHOD OF USE

(75) Inventors: Newton H. Metcalf, Memphis, TN (US); Greg C. Marik, Collierville, TN (US)

(73) Assignee: WARSAW ORTHOPEDIC, INC, Warsaw, IN (US)

(21) Appl. No.: 12/838,583

(22) Filed: Jul. 19, 2010

(51) Int. Cl. A61F 2/44

(52) U.S. Cl. 623/17.11

(57) ABSTRACT

A spinal implant includes a body defining a longitudinal axis and an outer surface. The outer surface includes a first portion configured to engage a first bone surface and a second portion configured to engage a second opposing bone surface. A first locking element has a center of rotation along a first axis offset from the longitudinal axis. The first locking element is rotatable relative to the body between a first, non-engaging configuration and a second engaging configuration such that the first locking element extends beyond the outer surface. Methods of use are disclosed.

### TECHNICAL FIELD

The present disclosure generally relates to medical devices, systems and methods for the treatment of musculoskeletal disorders, and more particularly to an implant system for treating a vertebral column, which includes an interbody implant having a rotatable locking element configured to enhance fixation with adjacent bone structures.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, discectomy, laminectomy and implantable prosthetics. Fusion and fixation treatments may employ implants such as interbody fusion devices to achieve arthrodesis. This disclosure describes an improvement over these prior art technologies.

### SUMMARY OF THE INVENTION

Accordingly, an implant system and method is provided for treating a vertebral column. It is contemplated that the implant system includes an interbody implant having a rotatable locking element configured to enhance fixation with adjacent bone structures. It is further contemplated that the implant system may be employed for an arthrodesis treatment.

In one particular embodiment, in accordance with the principles of the present disclosure, a spinal implant is provided. The spinal implant includes a body defining a longitudinal axis and an outer surface. The outer surface includes a first portion configured to engage a first bone surface and a second portion configured to engage a second opposing bone surface. A first locking element has a center of rotation along a first axis offset from the longitudinal axis. The first locking element is rotatable relative to the body between a first, non-engaging configuration and a second engaging configuration such that the first locking element extends beyond the outer surface.

In one embodiment, an interbody spinal implant is provided. The interbody spinal implant includes a body defining a longitudinal axis and an outer surface. The outer surface includes a first endplate surface and a second opposing endplate surface. A first locking element has a center of rotation along an axis offset from the longitudinal axis. The first locking element is rotatable relative to the body between a first, non-engaging configuration and a second deployed configuration such that the first locking element extends beyond the first endplate surface in a configuration to lock the body with an endplate. A second locking element has a center of rotation along the axis offset from the longitudinal axis. The second locking element is rotatable relative to the body between a first, non-engaging configuration and a second, deployed configuration such that the second locking element extends beyond the second endplate surface in a configuration to lock the body with an endplate.

In one embodiment, an intervertebral fusion implant is provided. The intervertebral fusion implant includes a cage defining a longitudinal axis and an outer surface. The outer surface includes a first endplate engaging surface and a second opposing endplate engaging surface. The cage defines a substantially rectangular cross-section area defining a perimeter. A first locking element has a spike extending therefrom. The first locking element has a center of rotation along an axis offset from the longitudinal axis. The first locking element is rotatable between a first, non-engaging configuration such that the first spike of the first locking element is disposed within the perimeter and a second deployed configuration such that the spike of the first locking element extends beyond the first endplate engaging surface in a configuration to lock the cage with an endplate. A second locking element has a spike extending therefrom. The second locking element has a center of rotation along the offset axis. The second locking element is rotatable between a first, non-engaging configuration such that the spike of the second locking element is disposed within the perimeter and a second deployed configuration such that the spike of the second locking element extends beyond the second endplate engaging surface to lock the cage with an endplate. The cage defines a bore. The first locking element is connected to the second locking element with a shaft extending therebetween and disposed within the bore.

### BRIEF DESCRIPTION OF THE DRAWINGS

The present disclosure will become more readily apparent from the specific description accompanied by the following drawings, in which:

FIG. 1 is a perspective view of one particular embodiment of a spinal implant in accordance with the principles of the present disclosure;

FIG. 2 is a side, cross section view of the spinal implant shown in FIG. 1;

FIG. 3 is a schematic view of the spinal implant shown in FIG. 1;

FIG. 4 is a schematic view of the spinal implant shown in FIG. 1;

FIG. 5 is a perspective view of the spinal implant shown in FIG. 1 with a section of a vertebral column;

FIG. 6 is a perspective view of the spinal implant shown in part phantom with the section of the vertebral column shown in FIG. 5;

FIG. 7 is a perspective view of one embodiment employing a plurality of the spinal implant shown with the section of the vertebral column shown in FIG. 6; and

FIG. 8 is a side, cross section view of one embodiment of the spinal implant shown in FIG. 1.

### DETAILED DESCRIPTION OF THE INVENTION

The exemplary embodiments of the implant system and related methods of use disclosed are discussed in terms of medical devices for the treatment of musculoskeletal disorders and more particularly, in terms of an implant system for treating a vertebral column, which includes an interbody implant having a rotatable locking element configured to enhance fixation with adjacent bone structures. It is envisioned that the implant system and methods of use disclosed provide attachment of an interbody cage implant to adjacent bone endplate surfaces via a rotational lock disposed with the implant. It is further envisioned that the implant includes a rotation mechanism disposed off axis to create off axis rotation of the lock and deployment of the lock above a face of the implant. It is contemplated that disposing the internal rotation mechanism at an angle relative to the implant body axis facilitates deployment of locking elements, such as, for example, fixation teeth, in a configuration for fixation with opposing bone surfaces, such as, for example, bone surfaces disposed in an inferior/superior and/or an anterior/posterior configuration to maximize areas of engagement. It is further contemplated that the locking elements may be deployed with the above configurations in a rectangular cage geometry to minimize the required access space for implantation.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed implant system may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “superior” and “inferior” are relative and used only in the context to the other, and are not necessarily “upper” and “lower”.

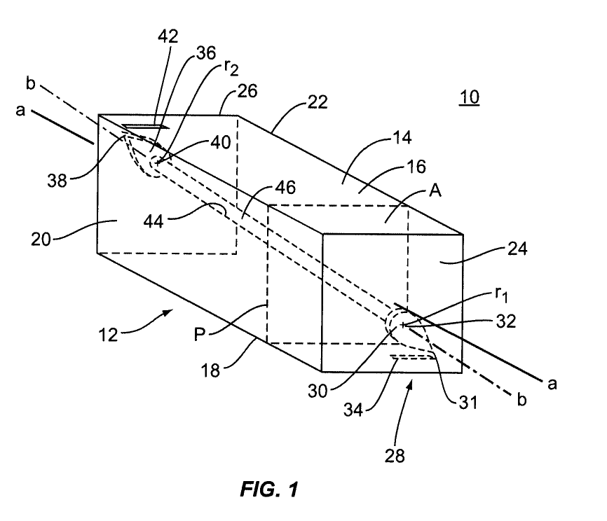

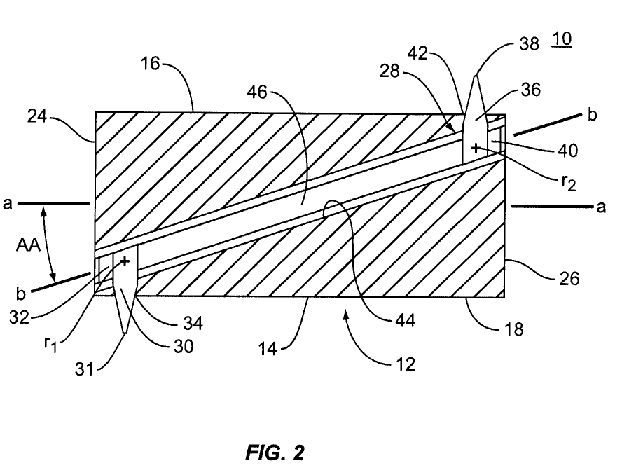

The following discussion includes a description of an implant system and related methods of employing the implant system in accordance with the principles of the present disclosure. Alternate embodiments are also disclosed. Reference will now be made in detail to the exemplary embodiments of the present disclosure, which are illustrated in the accompanying figures. Turning now to FIGS. 1-4, there is illustrated components of an implant system in accordance with the principles of the present disclosure.

The components of the implant system are fabricated from materials suitable for medical applications, including metals, polymers, ceramics, biocompatible materials and/or their composites, depending on the particular application and/or preference of a medical practitioner. For example, the components of the implant system, individually or collectively, can be fabricated from materials such as stainless steel, titanium, thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, biocompatible materials such as polymers including plastics, metals, ceramics and composites thereof, rigid polymers including polyphenylene, polyamide, polyimide, polyetherimide, polyethylene, epoxy, and various components of the implant system, may have material composites, including the above materials, to achieve various desired characteristics such as strength, rigidity, elasticity, compliance, biomechanical performance, durability and radiolucency or imaging preference.

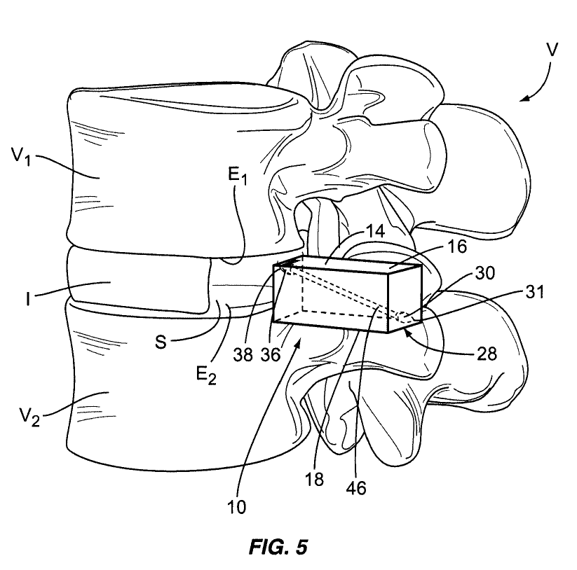

The implant system is configured for treating, for example, an affected section of a spine including vertebrae V, an intervertebral disc I and body areas adjacent thereto (FIG. 5). The implant system includes an interbody spinal implant 10 configured for disposal with a spinal column and adjacent areas within a body, such as, for example, an intervertebral space S between a first vertebrae V₁ and a second vertebrae V₂ of vertebrae V.

Implant 10 includes a body 12 having a cage configuration. The cage configuration of body 12 includes at least one cavity or openings (not shown) for disposal of bone growth material and/or other agent(s), as described below, for fusion and fixation applications of the implant system. It is contemplated that body 12 may have alternative configurations, such as, for example, solid, porous, multiple openings and/or passages therethrough.

Body 12 defines a longitudinal axis a and an outer surface 14. Outer surface 14 includes a first portion, such as, for example, an endplate engaging surface 16 configured to engage a first bone surface, such as, for example, a lower endplate E₁ of vertebrae V₁. Outer surface 14 includes a second portion, such as, for example, an endplate engaging surface 18 configured to engage an opposing, upper endplate E₂ of vertebrae V₂. It is envisioned that outer surface 14 engages opposing bone surfaces, which include bone surfaces that are oriented to face each other, oriented to face away from each other, oriented in transverse directions and/or oriented in alternate directions. It is further envisioned that surfaces 16, 18 may be smooth, arcuate, undulating and/or textured to enhance fixation with endplates E₁, E₂.

Body 12 has a substantially rectangular cross-sectional area A, which defines a perimeter P of body 12 along outer surface 14. Outer surface 14 includes lateral surfaces 20, 22 that define perimeter P of body 12 with surfaces 16, 18. Outer surface 14 also includes end surfaces 24, 26 to define a length of body 12. All or a portion of outer surface 14 may be variously configured such as, for example, smooth, arcuate, undulating and/or textured. It is contemplated that body 12 may have alternative cross sectional configurations such as, for example, circular, oval, polygonal, offset, staggered and/or tapered. Body 12 has a rectangular cross-section and the surfaces of outer surface 14 are substantially planar to facilitate disposal of implant 10 within intervertebral space S. It is envisioned that this configuration of implant 10 minimizes the access and/or space required for placement of implant 10 within a body space.

Implant 10 includes a lock 28 for attaching body 12 to adjacent bone structures, such as, for example, endplates E₁, E₂. Lock 28 is deployed for fixation with endplates E₁, E₂. Lock 28 includes a first locking element 30 having a center of rotation r₁ along a first axis b offset from longitudinal axis a. First axis b is offset at an angle AA from longitudinal axis a. It is envisioned that first axis b may alternatively by offset from longitudinal axis a in a parallel orientation.

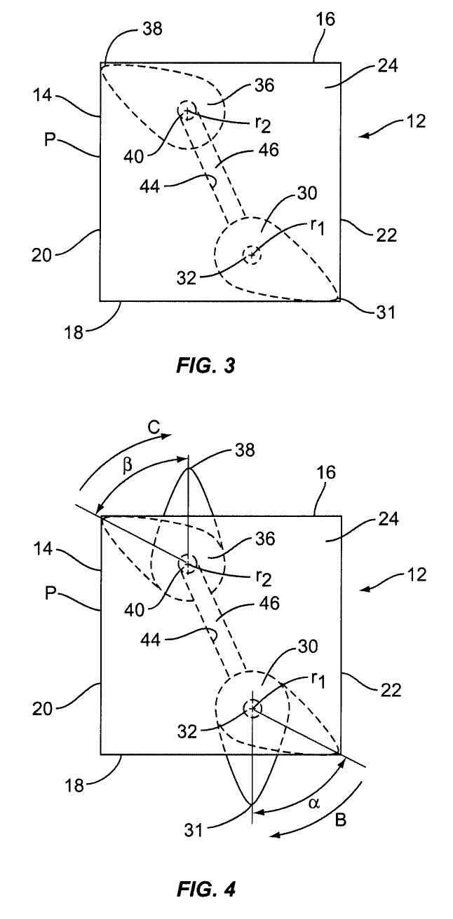

First locking element 30 is rotatable relative to body 12 between a first, non-engaging configuration (FIG. 3) and a second, engaging configuration (FIG. 4) such that at least a portion of first locking element 30 extends beyond outer surface 14. It is contemplated that the depth or length of locking element 30 that extends beyond outer surface 14 may be varied depending on the requirements of a particular application, such as, the depth of penetration into a bone or tissue surface and/or the accessible body space.

First locking element 30 includes a tooth or spike 31 configured to engage and penetrate adjacent bone structure, such as, for example, endplate E₂. It is envisioned that first locking element 30 may include one or a plurality of spikes 31. It is further envisioned that the bone engaging surface of first locking element 30 may be serrated, textured, staggered, uneven, undulating and/or smooth. It is contemplated that spike 31 may engage and catch or rest with a bone or tissue surface but not penetrate a bone or tissue surface.

First locking element 30 is mounted within body 12 with a pin 32 adjacent center of rotation r₁ for rotational movement of first locking element 30 relative to body 12. Body 12 defines a cavity, such as, for example, a slot 34 that allows first locking element 30 to rotate freely therein. Slot 34 extends such that first locking element 30 can rotate through an angle α, in the direction shown by arrow B in FIG. 4. It is contemplated that angle α is 45 degrees, however, may alternatively be in a range of 0-90 degrees. It is further contemplated that first locking element 30 may be rotated in a clockwise or counter-clockwise direction. It is envisioned that locking element 30 may also be axially movable relative to body 12, and may be mounted to outer surface 14 such that locking element 30 is disposed outside body 12.

Lock 28 includes a second locking element 36 having a center of rotation r₂ along first axis b, which is offset from longitudinal axis a. Second locking element 36 is rotatable relative to body 12 between a first, non-engaging configuration (FIG. 3) and a second, engaging configuration (FIG. 4) such that at least a portion of second locking element 36 extends beyond outer surface 14. It is contemplated that the depth or length of locking element 36 that extends beyond outer surface 14 may be varied depending on the requirements of a particular application, such as, the depth of penetration into a bone or tissue surface and/or the accessible body space.

Second locking element 36 includes a tooth or spike 38 configured to engage and penetrate adjacent bone structure, such as, for example, endplate E₁. It is envisioned that second locking element 36 may include one or a plurality spikes 38. It is further envisioned that the bone engaging surface of second locking element 36 may be serrated, textured, staggered, uneven, undulating and/or smooth. It is contemplated that spike 38 may engage and catch or rest with a bone or tissue surface but not penetrate a bone or tissue surface.

Second locking element 36 is mounted within body 12 with a pin 40 adjacent center of rotation r₂ for rotational movement of second locking element 36 relative to body 12. Body 12 defines a cavity, such as, for example, a slot 42 that allows second locking element 36 to rotate freely therein. Slot 42 extends such that second locking element 36 can rotate through an angle β, in the direction shown by arrow C in FIG. 4. It is contemplated that angle β is 45 degrees, however, may alternatively be in a range of 0-90 degrees. It is further contemplated that second locking element 36 may be rotated in a clockwise or counter-clockwise direction. It is envisioned that locking element 36 may also be axially movable relative to body 12, and may be mounted to outer surface 14 such that locking element 36 is disposed outside body 12.

Body 12 defines a bore 44 extending between slot 34 and slot 42. Bore 44 is configured for disposal of a shaft 46. Shaft 46 connects first locking element 30 with second locking element 36 for rotation relative to body 12. Shaft 46 is configured to rotate first locking element 30 and second locking element 36 simultaneously. Actuation of lock 28, which includes an assembly of locking elements 30, 36 and shaft 46, may include a mechanism internal to body 12, such as, for example, a spring biased element, pressure and/or a cavity, such as, for example, a socket, defined with one or both of elements 30, 36 for receiving a tool for rotating projections 30, 36. It is envisioned that body 12 may include a lever, button and/or clip that actuates shaft 46 to rotate locking elements 30, 36.

In assembly, operation and use, the implant system including implant 10 is employed with a surgical procedure for treatment of a spine of a patient including vertebrae V, intervertebral disc I and body areas adjacent thereto, as discussed herein. The implant system may also be employed with other surgical procedures, such as, for example, discectomy, laminectomy, fusion, laminotomy, laminectomy, nerve root retraction, foramenotomy, facetectomy, decompression, spinal nucleus or disc replacement and bone graft and implantable prosthetics including plates, rods, and bone engaging fasteners.

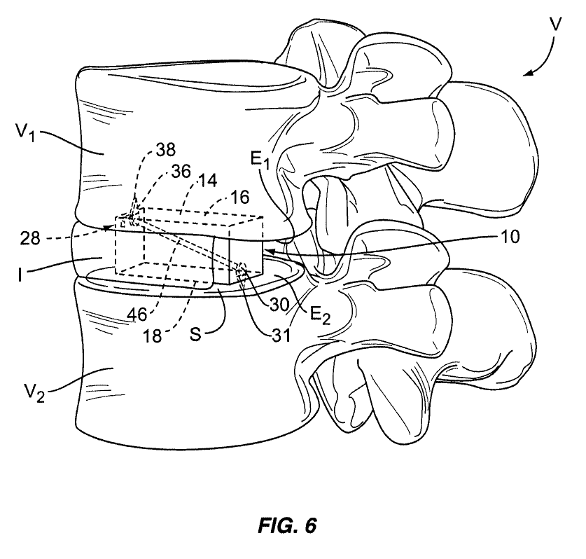

For example, as shown in FIGS. 5-6, the implant system is employed with a surgical arthrodesis procedure, such as, for example, fusion for treatment of an applicable condition or injury of an affected section of a spinal column and adjacent areas within a body, such as, for example, an intervertebral space S between a first vertebrae V₁ and a second vertebrae V₂ of vertebrae V. It is contemplated that the implant system is inserted with intervertebral space S to space apart articular joint surfaces, provide support and maximize stabilization of vertebrae V.

In use, to treat the affected section of vertebrae V, a medical practitioner obtains access to a surgical site including vertebrae V in any appropriate manner, such as through incision and retraction of tissues. It is envisioned that the implant system may be used in any existing surgical method or technique including open surgery, mini-open surgery, minimally invasive surgery and percutaneous surgical implantation, whereby vertebrae V is accessed through a mini-incision, or sleeve that provides a protected passageway to the area. Once access to the surgical site is obtained, the particular surgical procedure is performed for treating the spine disorder. The implant system is then employed to augment the surgical treatment. The implant system can be delivered or implanted as a pre-assembled device or can be assembled in situ. The implant system may be completely or partially revised, removed or replaced in situ. It is contemplated that one or all of the components of the implant system can be delivered to the surgical site via manual manipulation and/or a free hand technique.

A preparation instrument (not shown) is inserted within the protected passageway and disposed within intervertebral space S. The preparation instrument(s) are employed to remove disc tissue and fluids, adjacent tissues and/or bone, scrape and/or remove tissue from the surfaces of endplates E₁, E₂, as well as for aspiration and irrigation of the region according to the requirements of a particular surgical application.

Implant 10 is delivered to vertebrae V with a delivery instrument including a driver (not shown) via the protected passageway for the arthrodesis treatment. The driver delivers implant 10 into the prepared intervertebral space S, between vertebrae V₁ and vertebrae V₂, according to the requirements of a particular surgical application. Implant 10 is manipulated such that endplate engaging surface 16 engages endplate E₁ and endplate engaging surface 18 engages opposing endplate E₂.

Locking elements 30, 36 are initially disposed in the first, non-engaging configuration such that spikes 31, 38 are disposed within perimeter P along cross-section A of body 12, as shown in FIG. 3. In the first configuration, implant 10 is disposed in an insert and implant position. Spikes 31, 38 are recessed within slots 34, 42, respectively, such that body 12 can be implanted within intervertebral space S and do not interfere with implantation.

Shaft 46 and lock 28 are actuated and caused to rotate, as discussed, along axis b, which is offset from axis a. This off axis rotation facilitates positioning of locking elements 30, 36, which allow spikes 31, 38 to deploy into the second engaging configuration, as shown in FIG. 4, and endplates E₁, E₂. Locking element 30 rotates angle α and extends beyond perimeter P and endplate engaging surface 18, as shown by arrow B, into engagement and penetration with endplate E₂ in a configuration to lock body 12 with endplate E₂. Locking element 36 rotates angle β and extends beyond perimeter P and endplate engaging surface 16, as shown by arrow C, into engagement and penetration with endplate E₁ in a configuration to lock body 12 with endplate E₁.

The off axis rotation of shaft 46 and positioning of locking elements 30, 36 in the selected quadrants or corners of cross-section A allows spikes 31, 38 to rotate beyond the face of outer surface 14 and deploy outside of implant 10 for engagement with endplates E₁, E₂, thereby maximizing locking penetration with bony structures adjacent intervertebral space S. It is contemplated that spikes 31, 38 can deploy for locking engagement with respective interior and superior bone surfaces relative to outer surface 14. It is further contemplated that spikes 31, 38 can deploy for locking engagement with respective anterior and posterior bone surfaces relative to outer surface 14. This configuration facilitates locking engagement of implant 10 with both endplates E₁, E₂. It is envisioned that lock 28 locks implant 10 with one or a plurality of adjacent bone surfaces or structures.

Surfaces 16, 18 engage opposing endplates E₁, E₂, and lock 28 locks body 12 with endplates E₁, E₂ as discussed, such that implant 10 is secured within intervertebral space S to stabilize and immobilize vertebrae V. It is envisioned that the locking elements may additionally or alternatively include clips, hooks and/or flanges. It is contemplated that implant system 10 may be coated with an osteoconductive material such as hydroxyapatite and/or osteoinductive agent such as a bone morphogenic protein for enhanced bony fixation to the treated area. Implant system 10 can be made of radiolucent materials such as polymers. Radiomarkers may be included for identification under x-ray, fluoroscopy, CT or other imaging techniques.

In one embodiment, implant 10 may include voids and/or openings, for including therapeutic polynucleotides or polypeptides and bone growth promoting material, which can be packed or otherwise disposed therein. For example, such voids and/or openings may include at least one agent including biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as hydroxyapatite, calcium phosphate and calcium sulfite, biologically active agents, for example, biologically active agents coated onto the exterior of implant 10 and/or applied thereto for gradual release such as by blending in a bioresorbable polymer that releases the biologically active agent or agents in an appropriate time dependent fashion as the polymer degrades within the patient. Suitable biologically active agents include, for example, bone morphogenic protein (BMP) and cytokines.

Implant 10 may include one or a plurality of agent reservoirs. The agent reservoirs can be configured as drug depots with medication for pain and may include antibiotics and/or therapeutics. It is envisioned that the agent reservoirs contains active agents and may include one or a plurality of therapeutic agents and/or pharmacological agents for release, including sustained release, to treat, for example, pain, inflammation and degeneration. The agents may include pharmacological agents, such as, for example, antibiotics, anti-inflammatory drugs including but not limited to steroids, anti-viral and anti-retroviral compounds, therapeutic proteins or peptides, therapeutic nucleic acids (as naked plasmid or a component of an integrating or non-integrating gene therapy vector system), and combinations thereof.

The agent may also include analgesics or anesthetics such as acetic acid derivatives, COX-2 selective inhibitors, COX-2 inhibitors, enolic acid derivatives, propionic acid derivatives, salicylic acid derivatives, opioids, opioid/nonopioid combination products, adjuvant analgesics, and general and regional/local anesthetics.

The agent may also include antibiotics such as, for example, amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamthoxazole, and vancomycin.

The agent may also include immunosuppressives agents, such as, for example, steroids, cyclosporine, cyclosporine analogs, cyclophosphamide, methylprednisone, prednisone, azathioprine, FK-506, 15-deoxyspergualin, prednisolone, methotrexate, thalidomide, methoxsalen, rapamycin, leflunomide, mizoribine (Bredinin™), brequinar, deoxyspergualin, and azaspirane (SKF 105685), Orthoclone OKT™ 3 (muromonab-CD3). Sandimmune™, Neoral™, Sangdya™ (cyclosporine), Prograf™ (FK506, tacrolimus), Cellcept™ (mycophenolate motefil, of which the active metabolite is mycophenolic acid), Imuran™ (azathioprine), glucocorticosteroids, adrenocortical steroids such as Deltasone™ (prednisone) and Hydeltrasol™ (prednisolone), Folex™ and Mexate™ (methotrxate), Oxsoralen-Ultra™ (methoxsalen) and Rapamuen™ (sirolimus).

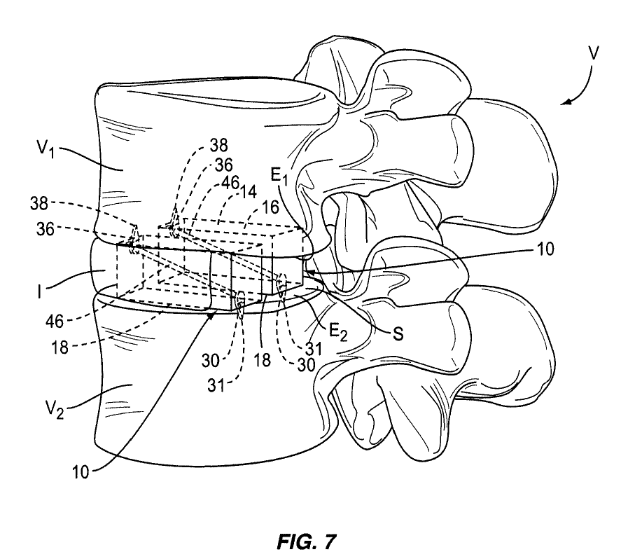

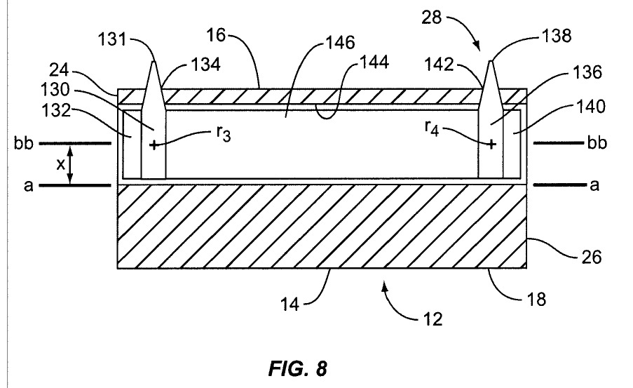

In one embodiment, as shown in FIG. 7, the implant system includes a plurality of implants 10, similar to that described above. A pair of implants 10 are disposed in a bilateral configuration within intervertebral space S between endplates E₁, E₂. It is contemplated that employing the plurality of implants 10 can optimize the amount intervertebral space S can be spaced apart such that the joint spacing dimension can be preselected. The plurality of implants 10 can be oriented in a side by side engagement, spaced apart, column and/or staggered.

In one embodiment, as shown in FIG. 8, implant 10, similar to that discussed above with regard to FIGS. 1-4, includes lock 28 having a first locking element 130 with a center of rotation r₃ along a first axis bb offset from longitudinal axis a. First axis bb is offset in parallel a distance X from longitudinal axis a. First locking element 130 is rotatable relative to body 12 between a first, non-engaging configuration and a second, engaging configuration, similar to that discussed above, such that at least a portion of first locking element 130 extends beyond outer surface 14. First locking element 130 includes a tooth or spike 131 configured to engage and penetrate endplate E₁ (FIGS. 5-6).

First locking element 130 is mounted within body 12 with a pin 132 adjacent center of rotation r₃ for rotational movement of first locking element 130 relative to body 12. Body 12 defines a slot 134 that allows first locking element 130 to rotate freely therein. Lock 28 includes a second locking element 136 having a center of rotation r₄ along first axis bb, which is offset from longitudinal axis a. Second locking element 136 is rotatable relative to body 12 between a first, non-engaging configuration and a second, engaging configuration, similar to that discussed, such that at least a portion of second locking element 136 extends beyond outer surface 14.

Second locking element 136 includes a tooth or spike 138 configured to engage and penetrate endplate E₁. Second locking element 136 is mounted within body 12 with a pin 140 adjacent center of rotation r₄ for rotational movement of second locking element 136 relative to body 12. Body 12 defines a slot 142 that allows second locking element 136 to rotate freely therein.

Body 12 defines a bore 144 extending between slot 134 and slot 142. Bore 144 is configured for disposal of a shaft 146. Shaft 146 connects first locking element 130 with second locking element 136 for rotation relative to body 12. Shaft 146 is configured to rotate first locking element 130 and second locking element 136 simultaneously.

Locking elements 130, 136 are initially disposed in the first, non-engaging configuration such that spikes 131, 138 are disposed within perimeter P along cross-section A of body 12. Spikes 131, 138 are recessed within slots 134, 142, respectively, such that body 12 can be implanted within intervertebral space S and do not interfere with implantation. Shaft 146 and lock 28 are actuated and caused to rotate along axis bb, which is offset from axis a. Locking elements 130, 136 and spikes 131, 138 deploy into the second engaging configuration and endplate E₁. Locking element 130 rotates and extends beyond perimeter P and endplate engaging surface 16 into engagement and penetration with endplate E₁ in a configuration to lock body 12 with endplate E₁. Locking element 136 rotates and extends beyond perimeter P and endplate engaging surface 16 into engagement and penetration with endplate E₁ in a configuration to lock body 12 with endplate E₁.

In one embodiment, implant 10, similar to that discussed above with regard to FIGS. 1-4, includes center of rotation r2 of second locking element 36 being defined along a second axis (not shown), which is offset from longitudinal axis a and independent of first axis b. Shaft 46 includes telescoping shafts and/or a bushing/bearing assembly to facilitate rotation of locking elements 30, 36 relative to body 12, as well as rotation of locking element 30 relative to locking element 36. First locking element 30 can be rotated selectively and independently from second locking element 36, and relative to body 12.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal implant comprising:
a body defining a longitudinal axis and an outer surface, the outer surface including a first portion configured to engage a first bone surface and a second portion configured to engage a second opposing bone surface; and
a first locking element having a center of rotation along a first axis offset from the longitudinal axis, the first locking element being rotatable relative to the body between a first, non-engaging configuration and a second engaging configuration such that the first locking element extends beyond the outer surface.

2. A spinal implant according to claim 1, wherein the first bone surface is a lower endplate of a superior vertebrae and the second bone surface is an upper endplate of an inferior vertebrae.

3. A spinal implant according to claim 1, wherein the body defines a cross-sectional area defining a perimeter such that first configuration includes the locking element being disposed within the perimeter and the second configuration includes the first locking element extending beyond the perimeter.

4. A spinal implant according to claim 1, wherein the body has a rectangular cross-section configuration.

5. A spinal implant according to claim 1, wherein the body defines a cross-sectional configuration including equal sides.

6. A spinal implant according to claim 1, further comprising a second locking element defining a center of rotation along a second axis offset from the longitudinal axis, the second locking element being rotatable between a first, non-engaging configuration and a second engaging configuration such that the second rotatable locking element extends beyond the outer surface.

7. A spinal implant according to claim 1, wherein the first locking element is rotatable through an angle of 45 degrees.

8. A spinal implant according to claim 1, further comprising a second locking element defining a center of rotation along the first axis, the first axis being disposed at an angle relative to the longitudinal axis, the second locking element being rotatable between a first, non-engaging configuration and a second engaging configuration such that the second locking element extends beyond the outer surface.

9. A spinal implant according to claim 8, wherein the body defines a bore, the first locking element being connected to the second locking element with a shaft extending therebetween and disposed within the bore.

10. A spinal implant according to claim 9, wherein the shaft is configured to rotate the first locking element and the second locking element simultaneously.

11. A spinal implant according to claim 9, wherein the shaft is configured to selectively and independently rotate the first locking element and the second locking element.

12. A spinal implant according to claim 1, further comprising a second locking element defining a center of rotation along the first axis, the second locking element being rotatable between a first, non-engaging configuration and a second non-engaging configuration such that the second locking element extends beyond the outer surface.

13. An interbody spinal implant comprising:
a body defining a longitudinal axis and an outer surface, the outer surface including a first endplate surface and a second opposing endplate surface;
a first locking element having a center of rotation along an axis offset from the longitudinal axis, the first locking element being rotatable relative to the body between a first, non-engaging configuration and a second deployed configuration such that the first locking element extends beyond the first endplate surface in a configuration to lock the body with an endplate; and
a second locking element having a center of rotation along the axis offset from the longitudinal axis, the second locking element being rotatable relative to the body between a first, non-engaging configuration and a second, deployed configuration such that the second locking element extends beyond the second endplate surface in a configuration to lock the body with an endplate.

14. An interbody spinal implant according to claim 13, wherein the body defines a cross-sectional area defining a perimeter such that the first configuration includes the first locking element and the second locking element being disposed within the perimeter and the second configuration includes the first locking element and the second locking element extending beyond the perimeter.

15. An interbody spinal implant according to claim 13, wherein the offset axis is disposed at an angle relative to the longitudinal axis.

16. An interbody spinal implant according to claim 15, wherein the body defines a bore, the first locking element being connected to the second locking element with a shaft extending therebetween and disposed within the bore.

17. An interbody spinal implant according to claim 16, wherein the shaft is configured to rotate the first locking element and the second locking element simultaneously.

18. An interbody spinal implant according to claim 16, wherein the shaft is configured to selectively and independently rotate the first locking element and the second locking element.

19. An interbody implant according to claim 13, wherein the body defines a cross-section configuration including equal sides.

20. An intervertebral fusion implant comprising:
a cage defining a longitudinal axis and an outer surface, the outer surface including a first endplate engaging surface and a second opposing endplate engaging surface, the cage defining a substantially rectangular cross-section area defining a perimeter;
a first locking element having a spike extending therefrom, the first locking element having a center of rotation along an axis offset from the longitudinal axis, the first locking element being rotatable between a first, non-engaging configuration such that the first spike of the first locking element is disposed within the perimeter and a second deployed configuration such that the spike of the first locking element extends beyond the first endplate engaging surface in a configuration to lock the cage with an endplate; and
a second locking element having a spike extending therefrom, the second locking element having a center of rotation along the offset axis, the second locking element being rotatable between a first, non-engaging configuration such that the spike of the second locking element is disposed within the perimeter and a second deployed configuration such that the spike of the second locking element extends beyond the second endplate engaging surface to lock the cage with an endplate,
wherein the cage defines a bore, the first locking element being connected to the second locking element with a shaft extending therebetween and disposed within the bore.
